(19) United States
(12) Patent Application Publication
(10) Pub. No.: US 2017/0065177 A1
(43) Pub. Date: Mar. 9, 2017

(54) SENSOR AND SENSOR SYSTEM

(71) Applicant: Kabushiki Kaisha Toshiba, Minato-ku (JP)

(72) Inventors: Keiji SUGI, Fujisawa (JP); Tomio ONO, Yokohama (JP); Toshiya YONEHARA, Kawasaki (JP)

(73) Assignee: Kabushiki Kaisha Toshiba, Minato-ku (JP)

(21) Appl. No.: 15/254,429

(22) Filed: Sep. 1, 2016

(30) Foreign Application Priority Data
Sep. 8, 2015 (JP) 2015-176656

(51) Int. Cl. A61B 5/00; H01L 27/28; A61B 5/02

(52) U.S. Cl. CPC A61B 5/0059; A61B 5/02007; H01L 27/288; A61B 2562/0233; A61B 2562/046; A61B 2576/00

(57) ABSTRACT

According to one embodiment, a sensor includes a plurality of light-emitting elements, and a light receiving element. The light-emitting elements are arranged along a first direction and a second direction, the second direction intersecting the first direction. At least a portion of light emitted from each of the light-emitting elements is incident on the light receiving element. The light-emitting elements do not overlap the light receiving element in a third direction, the third direction being perpendicular to the first direction and the second direction. The light-emitting elements include a first light-emitting element and a second light-emitting element. A position of the second light-emitting element in a plane is between a position of the first light-emitting element in the plane and a position of the light receiving element in the plane. The plane includes the first direction and the second direction.

### CROSS-REFERENCE TO RELATED APPLICATIONS

This application is based upon and claims the benefit of priority from Japanese Patent Application No. 2015-176656, filed on Sep. 8, 2015; the entire contents of which are incorporated herein by reference.

### FIELD

Embodiments described herein relate generally to a sensor and a sensor system.

### BACKGROUND

There is a sensor that irradiates light on a sensing object and senses the light passing through the sensing object. For example, the sensor is applied to the diagnosis of a living body, etc. High sensing precision is desirable for the sensor.

### BRIEF DESCRIPTION OF THE DRAWINGS

FIG. 1A and FIG. 1B are schematic views illustrating a sensor according to a first embodiment;

FIG. 2A and FIG. 2B are schematic plan views illustrating operations of the sensor according to the first embodiment;

FIG. 3 is a flowchart illustrating operations of the sensor according to the first embodiment;

FIG. 4A and FIG. 4B are schematic cross-sectional views illustrating a portion of the sensor according to the first embodiment;

FIG. 5A to FIG. 5C are schematic cross-sectional views illustrating other sensors according to the first embodiment;

FIG. 6A and FIG. 6B are schematic views illustrating another sensor according to the first embodiment;

FIG. 7A to FIG. 7C are schematic cross-sectional views illustrating other sensors according to the first embodiment;

FIG. 8A and FIG. 8B are schematic views illustrating a sensor according to the second embodiment;

FIG. 9A and FIG. 9B are schematic plan views illustrating operations of a sensor according to a second embodiment;

FIG. 10A and FIG. 10B are schematic plan views illustrating operations of the sensor according to the second embodiment;

FIG. 11A and FIG. 11B are schematic plan views illustrating other operations of the sensor according to the second embodiment;

FIG. 12A and FIG. 12B are schematic views illustrating another sensor according to the second embodiment; and

FIG. 13A and FIG. 13B are schematic views illustrating sensor systems according to a third embodiment.

### DETAILED DESCRIPTION

According to one embodiment, a sensor includes a plurality of light-emitting elements, and a light receiving element. The light-emitting elements are arranged along a first direction and a second direction, the second direction intersecting the first direction. At least a portion of light emitted from each of the light-emitting elements is incident on the light receiving element. The light-emitting elements do not overlap the light receiving element in a third direction, the third direction being perpendicular to the first direction and the second direction. The light-emitting elements include a first light-emitting element and a second light-emitting element. A position of the second light-emitting element in a plane is between a position of the first light-emitting element in the plane and a position of the light receiving element in the plane. The plane includes the first direction and the second direction.

According to another embodiment, a sensor includes a plurality of light-emitting elements and a plurality of light receiving elements. The light-emitting elements are arranged along a first direction and a second direction, the second direction intersecting the first direction. The light receiving elements are arranged along the first direction and the second direction. At least a portion of light emitted from each of the light-emitting elements is incident on the light receiving elements. The light-emitting elements and the light receiving elements are arranged alternately along the first direction.

Various embodiments will be described hereinafter with reference to the accompanying drawings.

The drawings are schematic and conceptual, and the relationships between the thickness and width of portions, the size ratio among portions, etc., are not necessarily the same as the actual values thereof. Further, the dimensions and proportions may be illustrated differently among drawings, even for identical portions.

In the present specification and drawings, the same elements as those described previously with reference to earlier figures are labeled with like reference numerals, and the detailed description thereof is omitted as appropriate.

First embodiment

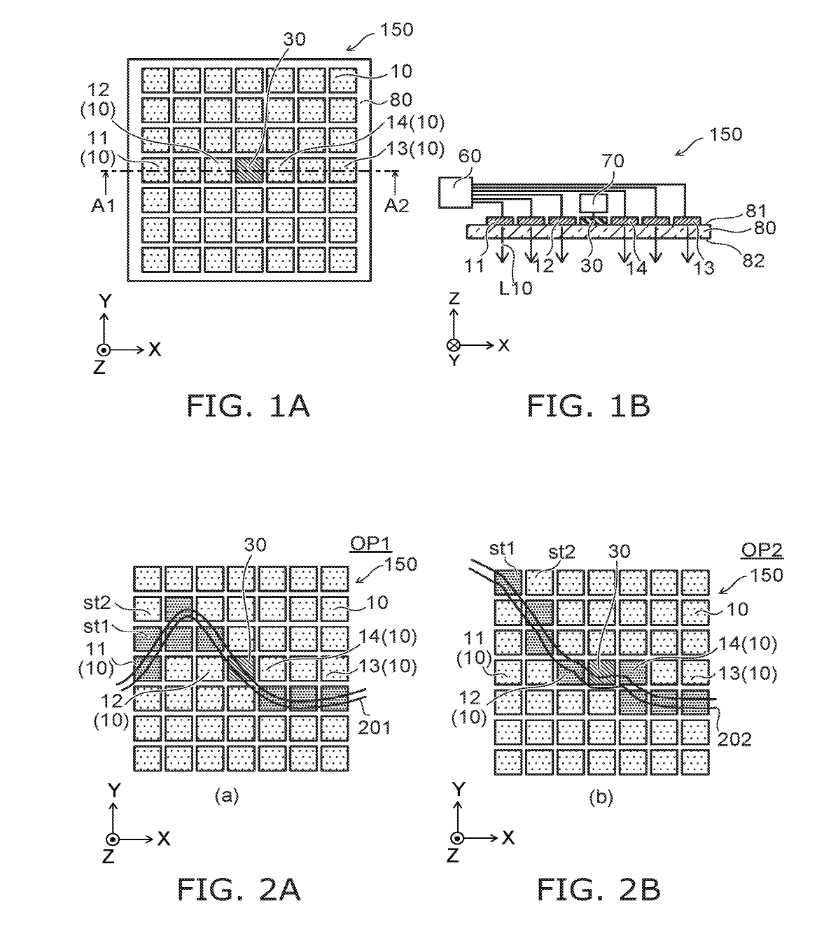

FIG. 1A and FIG. 1B are schematic views illustrating a sensor according to a first embodiment. FIG. 1A is a plan view. FIG. 1B is a line A1-A2 cross-sectional view of FIG. 1A.

As shown in FIG. 1A, the sensor 150 according to the embodiment includes a light receiving element 30 and multiple light-emitting elements 10.

The multiple light-emitting elements 10 are arranged along a first direction and a second direction. The second direction intersects the first direction. The first direction is taken as an X-axis direction. One axis perpendicular to the X-axis direction is taken as a Y-axis direction. A direction perpendicular to the X-axis direction and the Y-axis direction is taken as a Z-axis direction.

In the example, the second direction is perpendicular to the first direction. The second direction corresponds to the Y-axis direction. A direction perpendicular to the first direction and the second direction is taken as a third direction. The third direction is, for example, the Z-axis direction.

As shown in FIG. 1B, light L10 is emitted from each of the multiple light-emitting elements 10. In the example, the light L10 passes through a substrate 80. The light L10 is irradiated on the sensing object. The light that is irradiated passes through the sensing object. The light that is irradiated is reflected by the sensing object. Or, the light that is irradiated is scattered by the sensing object. The light that undergoes at least one of reflection or scattering travels toward the light receiving element 30. In the example, the light that undergoes the at least one of reflection or scattering passes through the substrate 80 and is incident on the light receiving element 30. In other words, at least a portion of the light L10 emitted from each of the multiple light-emitting elements 10 is incident on the light receiving element 30. The light receiving element 30 senses the light. The sensing object is sensed using the sense signal.

The multiple light-emitting elements 10 are arranged in a matrix configuration along the X-Y plane. A portion of the multiple light-emitting elements 10 is arranged in the X-axis direction (the first direction). A portion of the multiple light-emitting elements 10 is arranged in the Y-axis direction (the second direction). For example, the first direction is one of the row direction or the column direction. The second direction is the other of the row direction or the column direction.

The multiple light-emitting elements 10 do not overlap the light receiving element 30 in the third direction (the Z-axis direction). In the example, the multiple light-emitting elements 10 are provided around the light receiving element 30 in the X-Y plane (a plane including the first direction and the second direction).

As shown in FIG. 1B, the sensor 150 may further include a controller 60 and a processor 70. The controller 60 is connected to the multiple light-emitting elements 10. The controller 60 controls the operations of the multiple light-emitting elements 10. The processor 70 is connected to the light receiving element 30. The processor 70 processes the light reception signal obtained by the light receiving element 30. The controller 60 and the processor 70 are not shown in FIG. 1A.

In the example, the sensor 150 further includes a substrate 80. The substrate 80 has a first surface 81 and a second surface 82. The first surface 81 is along the first direction and the second direction. The second surface 82 is the surface on the side opposite to the first surface 81. For example, the first surface 81 is the upper surface; and the second surface 82 is the lower surface.

In the example, the multiple light-emitting elements 10 are provided at the first surface 81. The light receiving element 30 also is provided at the first surface 81. As described below, the light receiving element 30 may be provided at the second surface 82. In the embodiment, the surface where the multiple light-emitting elements 10 are provided is the first surface 81. In the embodiment, the first surface 81 may be the lower surface; and the second surface 82 may be the upper surface.

For example, the multiple light-emitting elements 10 include a first light-emitting element 11 and a second light-emitting element 12. The second light-emitting element 12 is positioned between the first light-emitting element 11 and the light receiving element 30. In other words, the position of the second light-emitting element 12 in the X-Y plane (the plane including the first direction and the second direction) is between the position of the first light-emitting element 11 in the X-Y plane and the position of the light receiving element 30 in the X-Y plane.

For example, the multiple light-emitting elements 10 further include a third light-emitting element 13 and a fourth light-emitting element 14. The fourth light-emitting element 14 is positioned between the third light-emitting element 13 and the light receiving element 30. In other words, the position of the fourth light-emitting element 14 in the X-Y plane is between the position of the third light-emitting element 13 in the X-Y plane and the position of the light receiving element 30 in the X-Y plane. In the example, the light receiving element 30 is disposed between the first light-emitting element 11 and the third light-emitting element 13.

Thus, in the sensor 150, the multiple light-emitting elements 10 that have mutually-different distances to the light receiving element 30 are provided. By using such multiple light-emitting elements 10, high-precision sensing can be performed.

For example, the light emitted from the first light-emitting element 11 which is distal to the light receiving element 30 is incident on the light receiving element 30 after passing through a distal position of the sensing object. On the other hand, the light emitted from the second light-emitting element 12 which is proximal to the light receiving element 30 is incident on the light receiving element 30 after passing through a proximal position of the sensing object. By using the multiple light-emitting elements 10 that have mutually-different distances from the light receiving element 30, the sensing can be implemented with high precision for each of multiple positions having mutually-different distances from the sensor 150.

For example, the sensor 150 according to the embodiment is used to sense a living body. The living body includes an object (e.g., a blood vessel, etc.). The sensor 150 is disposed at the vicinity of the surface of the living body. The light that is emitted from the multiple light-emitting elements 10 enters the living body. The light that enters is reflected (scattered) by the blood vessel, etc., inside the living body. The light that is reflected (scattered) is incident on the light receiving element 30. For example, the living body has a position inside the living body proximal to the surface (a shallow position), and a position inside the living body distal to the surface (a deep position). For example, there are cases where the density of the blood vessels at the shallow position is lower than the density of the blood vessels existing at the deep position. In such a case, for example, the sensing is performed by using the light of the first light-emitting element 11 that passes through the deep position having the high density. Thereby, the state of the blood vessels at a desired position can be sensed with high precision. In other words, the precision of the sensing can be increased by sensing the information of the position at the desired depth using the light-emitting element 10 corresponding to the depth.

For example, the second light-emitting element 12 does not emit light when the first light-emitting element 11 is emitting light. Thereby, the effects of positions at depths that are not the target depth are suppressed. Thereby, the state of the object (the blood vessel) at the position of the target depth can be sensed with high precision.

Conversely, for example, the second light-emitting element 12 is caused to emit light; and the first light-emitting element 11 is caused to not emit light. Sensing is performed using the light of the second light-emitting element 12. Thereby, the state of the blood vessel at the shallow position can be sensed with high precision. In such a case, the effects of the deep position are suppressed because the first light-emitting element 11 does not emit light. Thereby, the state of the object (the blood vessel) at the shallow position which is the target can be sensed with high precision.

On the other hand, for example, there is a first reference example in which the distances are the same between the light receiving element and the multiple light-emitting elements. In the reference example, the multiple light-emitting elements are arranged on a concentric circle around the light receiving element. In the reference example, because the distances are the same between the light receiving element and the multiple light-emitting elements, it is difficult to obtain the information at positions of different depths with high precision.

In the embodiment, the second light-emitting element 12 is provided between the first light-emitting element 11 and the light receiving element 30. In other words, in one direction, the first light-emitting element 11 and the second light-emitting element 12 are provided at multiple positions having mutually-different distances from the light receiving element 30. By using such a first light-emitting element 11 and such a second light-emitting element 12, the information at positions of different depths can be obtained with high precision.

There are cases where the state of a designated blood vessel is examined as an examination of the living body. The position of the blood vessel from the surface of the living body may be different between the living bodies. For example, the thickness of layers of fat at the surface vicinity of the skin, etc., are different between living bodies. In such a case, the depth from the surface of the designated blood vessel is different between the living bodies. Even in such a case, by using the light-emitting elements corresponding to the different depths, the state of the designated blood vessel can be sensed with high precision.

There are also cases where the examination of the living body is performed continuously and constantly. In such a case, the examination is performed for a designated position inside the living body to reduce the burden. There are cases where the density of the blood vessels is low at the designated position. In such a case, sensing with high precision is possible by selectively sensing a region along the configuration of the blood vessel.

The multiple light-emitting elements 10 are arranged in a matrix configuration in the sensor 150. Therefore, the light L10 can be irradiated selectively on a region having any configuration. An example of such an operation will now be described.

FIG. 2A and FIG. 2B are schematic plan views illustrating operations of the sensor according to the first embodiment.

FIG. 2A illustrates a first operation OP1 implemented by the sensor 150. FIG. 2B illustrates a second operation OP2 implemented by the sensor 150. The substrate 80, the controller 60, the processor 70, etc., are not shown in these drawings.

In the first operation OP1 as shown in FIG. 2A, a blood vessel 201 is the object of the sensing. In such a case, a portion of the multiple light-emitting elements 10 is set to a first state st1 (a light-emitting state). Another portion of the multiple light-emitting elements 10 is set to a second state st2 (a non-light-emitting state). The light-emitting elements 10 in the first state st1 are arranged along the configuration of the blood vessel 201. For example, the light-emitting elements 10 that overlap the blood vessel 201 in the Z-axis direction are set to the first state st1. For example, the light-emitting elements 10 for which the distances to the blood vessel 201 are a prescribed threshold or more are set to the first state st1. The other light-emitting elements 10 are set to the second state st2.

In the non-light-emitting state (the second state st2), the light is not emitted from the light-emitting element 10. Or, in the non-light-emitting state (the second state st2), the intensity of the light emitted from the light-emitting element 10 is lower than the intensity of the light emitted from the light-emitting element 10 in the light-emitting state (the first state st1). For example, the intensity of the light in the non-light-emitting state is not more than 1/10 of the intensity of the light in the light-emitting state.

Thus, the state of the blood vessel 201 can be sensed with high precision by selectively setting the light-emitting elements 10 proximal to the blood vessel 201 to the light-emitting state and selectively setting the light-emitting elements 10 distal to the blood vessel 201 to the non-light-emitting state.

For example, when sensing the blood vessel 201 by setting the multiple light-emitting elements 10 to the light-emitting state regardless of the configuration of the blood vessel 201 when sensing, the information of regions that are not the blood vessel 201 are sensed together. The information of the regions that are not the blood vessel 201 becomes noise in the sensing.

In the embodiment, for example, the information of the blood vessel 201 can be sensed with high precision by setting, to the first state st1, the light-emitting elements 10 arranged along the configuration of the blood vessel 201.

In the second operation OP2 as shown in FIG. 2B, another blood vessel 202 is the object of the sensing. In such a case as well, a portion of the multiple light-emitting elements is set to the first state st1 (the light-emitting state). Another portion of the multiple light-emitting elements 10 is set to the second state st2 (the non-light-emitting state). The light-emitting elements 10 in the first state st1 are arranged along the configuration of the blood vessel 202. In the second operation OP2 as well, the information of the blood vessel 202 can be sensed with high precision by setting the light-emitting elements 10 along the configuration of the blood vessel 202 to the first state st1.

Such an operation can be implemented by the controller 60. For example, the controller 60 implements the first operation OP1 and the second operation OP2. For example, in the first operation OP1, the first light-emitting element 11 is in the first state st1 (a first light-emitting state); and the second light-emitting element 12 is in the second state st2 (a second non-light-emitting state). In the second operation OP2, the first light-emitting element 11 is in the second state st2 (a first non-light-emitting state); and the second light-emitting element 12 is in the first state st1 (a second light-emitting state).

In the first operation OP1 of the example of FIG. 2A, the third light-emitting element 13 is in the second state st2; and the fourth light-emitting element 14 is in the second state st2. In the second operation OP2 of the example of FIG. 2B, the third light-emitting element 13 is in the second state st2; and the fourth light-emitting element 14 is in the first state st1.

In the description recited above, the first light-emitting element 11 and the second light-emitting element 12 may be any two of the multiple light-emitting elements 10. In the description recited above, the third light-emitting element 13 and the fourth light-emitting element 14 may be any two of the multiple light-emitting elements 10.

For example, in FIG. 2A and FIG. 2B, the third light-emitting element 13 may be considered to be the first light-emitting element; and the fourth light-emitting element 14 may be considered to be the second light-emitting element. In such a case, in the first operation OP1, the first light-emitting element is in the second state st2 (the non-light-emitting state); and the second light-emitting element also is in the second state st2 (the non-light-emitting state). In the second operation OP2, the first light-emitting element is in the second state st2 (the non-light-emitting state); and the second light-emitting element is in the first state st1 (the light-emitting state). In other words, in the embodiment, at least one of the multiple light-emitting elements 10 may be in the light-emitting state or the non-light-emitting state.

For example, such operations may be performed by implementing a pre-operation. For example, the pre-operation is implemented prior to the sensing of the object.

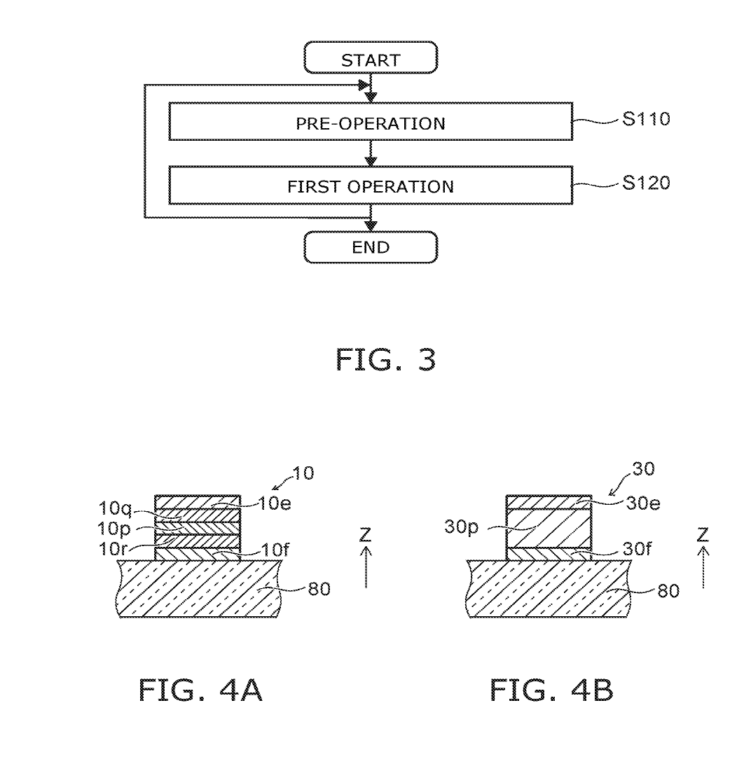

FIG. 3 is a flowchart illustrating operations of the sensor according to the first embodiment.

As shown in FIG. 3, the controller 60 implements a pre-operation (step S110). The pre-operation is, for example, a preparation operation. In the pre-operation, each of the multiple light-emitting elements 10 is set to the light-emitting state (the first state st1). For example, the multiple light-emitting elements 10 are set in order one at a time to the light-emitting state; and the light is sensed by the light receiving element 30 synchronously with the setting. For example, the state of the light is sensed for each of the multiple light-emitting elements 10 when the light-emitting element 10 is in the light-emitting state and the other light-emitting elements 10 are in the non-light-emitting state.

For example, in the case where the object of the sensing is a blood vessel, the change (the signal) of the light has a pulse wave shape. The interval in which the amplitude of the pulse wave shape is large is designated; and the light-emitting elements 10 that are set to the light-emitting state in the interval are designated. For example, a threshold that relates to the amplitude of the pulse wave shape is determined; and the sensed amplitude is compared to the threshold. The interval in which the amplitude of the pulse wave shape is large is designated based on the comparison result. Thus, by the pre-operation, the light-emitting elements 10 for which high sensitivity is obtained for the target object (the blood vessel) are determined. In other words, the first light-emitting element 11 is determined from among the multiple light-emitting elements 10 based on the result of the pre-operation. The second light-emitting element 12 is determined from among the multiple light-emitting elements 10 based on the result of the pre-operation. Based on the result of the pre-operation, the light-emitting elements 10 to be set to the light-emitting state are determined; and the other light-emitting elements 10 are set to the non-light-emitting state.

Then, after the pre-operation, the controller 60 implements the first operation OP1 (step S120). In the first operation OP1, the first light-emitting element 11 that is determined based on the result of the pre-operation is in the first state st1 (the first light-emitting state); and the second light-emitting element 12 that is determined based on the result of the pre-operation is in the second state st2 (the second non-light-emitting state).

In other words, by the pre-operation, the light-emitting elements 10 that correspond to the configuration of the object of the sensing (e.g., the blood vessel or the like), etc., are designated; and the light-emitting elements 10 that are designated are set to the light-emitting state. The other light-emitting elements 10 are set to the non-light-emitting state. Thereby, one object can be sensed with high precision.

As shown in FIG. 3, such operations may be further implemented repeatedly. In other words, the pre-operation is implemented again after implementing the first operation OP1. Another first operation (i.e., the second operation OP2) may be implemented based on the result of the pre-operation that is implemented again. The second operation OP2 is implemented for the case of a different object, for the case where the relative positions of the sensor 150 and the object have moved, etc. In the second operation OP2 as well, high-precision sensing is performed.

In the sensor 150 according to the embodiment as recited above, the multiple light-emitting elements 10 are arranged in a matrix configuration. A portion of the multiple light-emitting elements 10 is set to the light-emitting state according to the configuration of the object of the examination; and the other light-emitting elements 10 are set to the non-light-emitting state. Thereby, high-precision sensing is possible.

On the other hand, there is a second reference example in which one light-emitting element and multiple sensing elements are provided. In the reference example, an imaging image is acquired by the multiple sensing elements; the position (the depth, etc.) of the blood vessel is designated based on the imaging image; and based on the result, for example, the components inside the blood vessel are sensed. In the reference example, complex image processing is performed; and the device (particularly, the processing circuit) is complex. Further, the second reference example is expensive because the multiple light receiving elements are arranged in a matrix configuration.

Further, in the second reference example, the light that is emitted from the one light-emitting element spreads over a wide range; and the imaging is performed using the light passing through the wide range. The light is scattered by the multiple objects (the blood vessels, etc.) existing in the wide range and is incident on the multiple light receiving elements. The light that is reflected (scattered) by each of the multiple objects also is incident on other objects and is further reflected (scattered). Therefore, the light that passes through the wide range is affected by the multiple objects. Therefore, in the second reference example, it is difficult to sense the designated object with high precision.

In the case where the object is a blood vessel, etc., the signal that is sensed has a pulse wave shape. The pulse wave changes temporally and is substantially periodic. In the case where such a pulse wave is generated by multiple objects within a wide range, a time delay of the pulse wave occurs due to different positions within the wide range. In other words, a temporal shift is superimposed onto the pulse wave according to the different positions of the objects (the blood vessels, etc.) existing in the wide range. The light is affected by the superimposition of the temporal shift. Such light reaches the multiple light receiving elements. Therefore, in the second reference example, high-precision sensing is difficult due to the effect of the temporal shift of the pulse wave.

Conversely, in the sensor 150 according to the embodiment, the effect of the temporal shift of the pulse wave is suppressed by selectively setting the light-emitting elements 10 corresponding to the target object (the blood vessel, etc.) to the light-emitting state. Therefore, high-precision sensing is possible also when sensing the object of the pulse wave.

In the embodiment, complex image processing and the like are unnecessary; and the device is simple. Therefore, the cost can be reduced.

On the other hand, there is a third reference example in which a light receiving element overlaps multiple light-emitting elements in the Z-axis direction. At least a portion of the multiple light-emitting elements overlaps the light receiving element in the Z-axis direction. In such a case, a portion of the light receiving surface of the light receiving element is shielded by the light-emitting elements. Therefore, the light receiving element cannot be utilized sufficiently; and the sensing efficiency is insufficient. Therefore, in the third reference example, the increase of the sensing sensitivity is insufficient.

Conversely, in the embodiment, the multiple light-emitting elements 10 do not overlap the light receiving element 30 in the Z-axis direction. Therefore, the light receiving surface of the light receiving element 30 is not shielded by the light-emitting elements 10. The sensing efficiency is high because the light receiving element 30 can be utilized sufficiently. In the embodiment, the sensing sensitivity can be increased. Thereby, high-precision sensing is possible.

In the embodiment, the noise of the sensing can be reduced by using organic light-emitting layers as the multiple light-emitting elements 10. According to investigations by the inventor, it was found that the noise of the light radiated from light-emitting elements including organic light-emitting layers is lower than the noise of the light radiated from light-emitting elements including inorganic light-emitting layers (e.g., a semiconductor crystal, etc.). For example, in the semiconductor crystal, the light is emitted by carriers recombining with a constant probability. It is considered that the noise corresponding to the fluctuation of the recombination in the semiconductor crystal occurs because the fluctuation of the recombination is relatively small.

Conversely, in the organic light-emitting layer, it is considered that the recombination is averaged temporally because the fluctuation of the compound included in the organic light-emitting layer is large. As a result, it is considered that the noise is low in the case where the organic light-emitting layer is used. In particular, in the case where the signal having the pulse wave shape is sensed, the pulse wave can be sensed with high precision by stably sensing the temporal change of the signal. By using light having low noise, the pulse wave can be sensed with high precision. The light that is radiated from the light-emitting elements including organic light-emitting layers is suited to applications that sense a sensing object (e.g., a pulse wave, etc.) outputting a micro signal.

In the embodiment, the multiple light-emitting elements 10 are provided; and the multiple light-emitting elements 10 are selectively set to the light-emitting state. Therefore, the driving time of each of the multiple light-emitting elements 10 is short. The driving life may be shorter for an organic light-emitting layer than for an inorganic light-emitting layer (e.g., a semiconductor crystal, etc.). In the embodiment, the driving time of each of the multiple light-emitting elements 10 is short when the multiple light-emitting elements 10 include organic light-emitting layers; therefore, the problem of the shortness of the driving life of the organic light-emitting layer is reduced.

FIG. 4A and FIG. 4B are schematic cross-sectional views illustrating a portion of the sensor according to the first embodiment.

FIG. 4A illustrates one of the multiple light-emitting elements 10. FIG. 4B illustrates the light receiving element 30.

As shown in FIG. 4A, the light-emitting element 10 includes a first electrode 10e, a second electrode 10f, an organic light-emitting layer 10p, a first intermediate layer 10q, and a second intermediate layer 10r. The organic light-emitting layer 10p is disposed between the first electrode 10e and the second electrode 10f. The first intermediate layer 10q is disposed between the first electrode 10e and the organic light-emitting layer 10p. The second intermediate layer 10r is disposed between the second electrode 10f and the organic light-emitting layer 10p. In the example, the second electrode 10f is disposed between the first electrode 10e and the substrate 80. In the embodiment, the first electrode 10e may be disposed between the second electrode 10f and the substrate 80.

A current is caused to flow in the organic light-emitting layer 10p via the first intermediate layer 10q and the second intermediate layer 10r by applying a voltage between the first electrode 10e and the second electrode 10f. Thereby, the light L10 is emitted from the organic light-emitting layer 10p. The light L10 that is emitted is emitted to the outside via one of the first electrode 10e or the second electrode 10f.

For example, the reflectance of the first electrode 10e is higher than the reflectance of the second electrode 10f. For example, the transmittance of the second electrode 10f is higher than the transmittance of the first electrode 10e. In such a case, the light L10 is emitted to the outside via the second electrode 10f. The light L10 further passes through the substrate 80. The light that is reflected (scattered) by the sensing object is incident on the light receiving element 30 after passing through the substrate 80. In the example, the light-emitting element 10 is a bottom-emission type. In the embodiment, the light-emitting element 10 may be a top-emission type.

The first electrode 10e includes, for example, at least one of aluminum, silver, or gold. The first electrode 10e may include, for example, an alloy of magnesium and silver.

The second electrode 10f includes, for example, ITO (Indium Tin Oxide). The second electrode 10f may include, for example, a conductive polymer such as PEDOT:PSS, etc. The second electrode 10f may include, for example, a metal (e.g., aluminum, silver, etc.). In the case where the second electrode 10f includes a metal, it is favorable for the thickness of the second electrode 10f to be not less than 5 nm (nanometers) and not more than 20 nm. Thereby, light transmissivity is obtained.

The first intermediate layer 10q includes, for example, at least one of Alq₃, BAlq, POPy₂, Bphen, or 3TPYMB. For example, the first intermediate layer 10q functions as an electron transport layer. The first intermediate layer 10q may include, for example, at least one of LiF, CsF, Ba, or Ca. For example, the first intermediate layer 10q may function as an electron injection layer.

The organic light-emitting layer 10p includes, for example, at least one of Alq₃ (tris(8-hydroxyquinolinato)aluminum), F8BT(poly(9,9-dioctylfluorene-co-benzothiadiazole), or PPV (polyparaphenylene vinylene).

The organic light-emitting layer 10p may include a mixed material containing a host material and a dopant added to the host material.

The host material includes, for example, at least one of CBP (4,4′-N,N′-bis dicarbazolyl-biphenyl), BCP (2,9-dimethyl-4,7 diphenyl-1,10-phenanthroline), TPD (-dimethyl-4,7 diphenyl-1,10-phenanthroline), PVK (polyvinyl carbazole), or PPT (poly(3-phenylthiophene)).

The dopant material includes, for example, at least one of Flrpic(iridium(III)-bis(4,6-di-fluorophenyl)-pyridinate-N,C2′-picolinate), Ir(ppy)₃ (tris(2-phenylpyridine)iridium), or Flr6 (bis(2,4-difluorophenylpyridinato)-tetrakis(1-pyrazolyl)borate-iridium(III)).

The second intermediate layer 10r includes, for example, at least one of α-NPD, TAPC, m-MTDATA, TPD, or TCTA. For example, the second intermediate layer 10r functions as a hole transport layer. The second intermediate layer 10r may include, for example, at least one of PEDPOT:PPS, CuPc, or MoO₃. The second intermediate layer 10r may function as a hole injection layer.

The substrate 80 includes, for example, glass. The thickness of the substrate 80 is, for example, not less than 0.05 mm and not more than 2.0 mm. The substrate 80 may include a resin.

As shown in FIG. 4B, the light receiving element 30 includes a third electrode 30e, a fourth electrode 30f, and a photoelectric conversion layer 30p. The photoelectric conversion layer 30p is provided between the third electrode 30e and the fourth electrode 30f. In the example, the fourth electrode 30f is disposed between the third electrode 30e and the substrate 80. In the embodiment, the third electrode 30e may be disposed between the fourth electrode 30f and the substrate 80. For example, the photoelectric conversion layer 30p is an organic photoelectric conversion layer. The photoelectric conversion layer 30p may include at least a portion of the material included in the organic light-emitting layer 10p. For example, at least a portion of the photoelectric conversion layer 30p may be formed when forming the organic light-emitting layer 10p. Thereby, processes can be omitted; and, for example, the cost can be reduced.

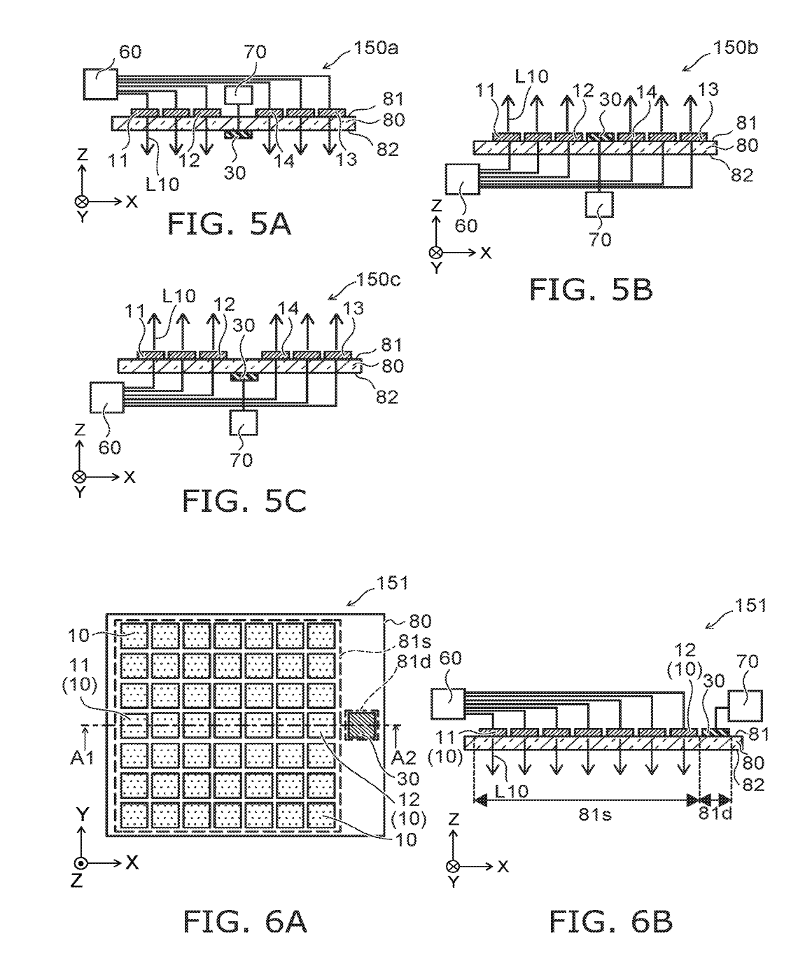

FIG. 5A to FIG. 5C are schematic cross-sectional views illustrating other sensors according to the first embodiment.

In a sensor 150a according to the embodiment as shown in FIG. 5A, the multiple light-emitting elements 10 are provided at the first surface 81 of the substrate 80; and the light receiving element 30 is provided at the second surface 82 of the substrate 80. The light L10 that is emitted from the multiple light-emitting elements 10 is incident on the sensing object after passing through the substrate 80. The light that is reflected (scattered) by the sensing object is incident on the light receiving element 30.

In a sensor 150b according to the embodiment as shown in FIG. 5B, the multiple light-emitting elements 10 and the light receiving element 30 are provided at the first surface 81 of the substrate 80. The light L10 that is emitted from the multiple light-emitting elements 10 is incident on the sensing object without passing through the substrate 80. The light that is reflected (scattered) by the sensing object is incident on the light receiving element 30.

In a sensor 150c according to the embodiment as shown in FIG. 5C, the multiple light-emitting elements 10 are provided at the first surface 81 of the substrate 80; and the light receiving element 30 is provided at the second surface 82 of the substrate 80. The light L10 that is emitted from the multiple light-emitting elements 10 is incident on the sensing object without passing through the substrate 80. The light that is reflected (scattered) by the sensing object is incident on the light receiving element 30 after passing through the substrate 80.

FIG. 6A and FIG. 6B are schematic views illustrating another sensor according to the first embodiment.

FIG. 6A is a plan view. FIG. 6B is a line A1-A2 cross-sectional view of FIG. 1A.

The multiple light-emitting elements 10 and the light receiving element 30 are included in the sensor 151 according to the embodiment as shown in FIG. 6A. In the example as well, the multiple light-emitting elements 10 are arranged along the first direction (the X-axis direction) and along the second direction (the Y-axis direction) intersecting the first direction. At least a portion of the light L10 emitted from each of the multiple light-emitting elements 10 is incident on the light receiving element 30. The arrangement of the multiple light-emitting elements 10 and the light receiving element 30 in the sensor 151 is different from that of the sensor 150. Otherwise, the sensor 151 is similar to the sensor 150. The arrangement of the multiple light-emitting elements 10 and the light receiving element 30 of the sensor 151 will now be described.

In the sensor 151, the light receiving element 30 is provided outside the region where the multiple light-emitting elements 10 are provided. In other words, the sensor 151 includes the substrate 80. The substrate 80 has the first surface 81 along the first direction and the second direction. The first surface 81 includes a first region 81s and a second region 81d. The second region 81d is positioned on the outer side of the first region 81s. The multiple light-emitting elements 10 overlap the first region 81s in the Z-axis direction (the third direction perpendicular to the first direction and the second direction). In other words, the multiple light-emitting elements 10 are provided in the first region 81s. On the other hand, the light receiving element 30 overlaps the second region 81d in the Z-axis direction (in the third direction). In other words, in the sensor 151, the light receiving element 30 is provided in a region (the second region 81d) on the outer side of the region (the first region 81s) where the multiple light-emitting elements 10 are provided.

In the sensor 151 as well, the multiple light-emitting elements 10 do not overlap the light receiving element 30 in the Z-axis direction. Thereby, because the light receiving surface of the light receiving element 30 is not shielded by the light-emitting elements 10, the sensing efficiency is high; and the sensing sensitivity can be increased.

In the sensor 151, the multiple light-emitting elements 10 include the first light-emitting element 11 and the second light-emitting element 12; and the position of the second light-emitting element 12 in the X-Y plane (a plane including the first direction and the second direction) is between the position of the first light-emitting element 11 in the X-Y plane and the position of the light receiving element 30 in the X-Y plane. The first light-emitting element 11 and the second light-emitting element 12 are provided at multiple positions having mutually-different distances from the light receiving element 30. Therefore, the information at positions of different depths can be obtained with high precision.

In the sensor 151 as well, the multiple light-emitting elements 10 are arranged in a matrix configuration. The light-emitting elements 10 that correspond to the target object (the blood vessel, etc.) are selectively set to the light-emitting state. For example, the effect of the temporal shift of the pulse wave is suppressed. High-precision sensing is possible when sensing the object of the pulse wave. High-precision sensing is possible by setting a portion of the multiple light-emitting elements 10 to the light-emitting state according to the configuration of the object of the examination and by setting the other light-emitting elements 10 to the non-light-emitting state.

In the sensor 151, the substrate 80 has the first surface 81, and the second surface 82 on the side opposite to the first surface 81. The multiple light-emitting elements 10 are provided at the first surface 81. In the sensor 151, the light receiving element 30 is provided at the first surface 81.

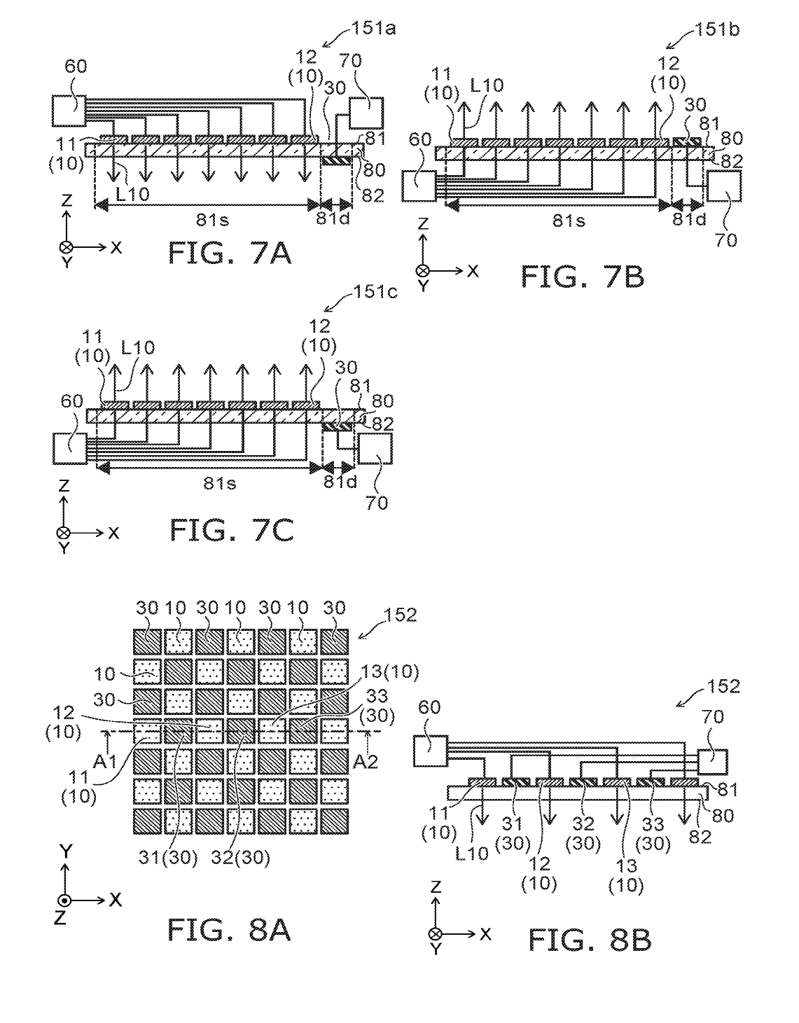

FIG. 7A to FIG. 7C are schematic cross-sectional views illustrating other sensors according to the first embodiment.

In a sensor 151a according to the embodiment as shown in FIG. 7A, the multiple light-emitting elements 10 are provided at the first surface 81 of the substrate 80; and the light receiving element 30 is provided at the second surface 82 of the substrate 80. The light L10 that is emitted from the multiple light-emitting elements 10 is incident on the sensing object after passing through the substrate 80. The light that is reflected (scattered) by the sensing object is incident on the light receiving element 30.

In a sensor 151b according to the embodiment as shown in FIG. 7B, the multiple light-emitting elements 10 and the light receiving element 30 are provided at the first surface 81 of the substrate 80. The light L10 that is emitted from the multiple light-emitting elements 10 is incident on the sensing object without passing through the substrate 80. The light that is reflected (scattered) by the sensing object is incident on the light receiving element 30.

In a sensor 151c according to the embodiment as shown in FIG. 7C, the multiple light-emitting elements 10 are provided at the first surface 81 of the substrate 80; and the light receiving element 30 is provided at the second surface 82 of the substrate 80. The light L10 that is emitted from the multiple light-emitting elements 10 is incident on the sensing object without passing through the substrate 80. The light that is reflected (scattered) by the sensing object is incident on the light receiving element 30 after passing through the substrate 80.

In the embodiment, the multiple light-emitting elements may have mutually-different peak wavelengths. The intensity of the light L10 emitted from the light-emitting element 10 has a maximum at the peak wavelength. For example, one of the multiple light-emitting elements 10 has a first peak wavelength. For example, one other of the multiple light-emitting elements 10 has a second peak wavelength.

The object of the sensing (e.g., the blood vessel) is sensed using two or more light-emitting elements 10 having different peak wavelengths. For example, the wavelength that has a high absorptance in the blood is different between different oxygen concentrations in the blood. By sensing the blood vessel using two or more light-emitting elements 10 having different peak wavelengths, for example, information relating to the oxygen concentration in the blood can be obtained. The applications of the sensor are expanded by using light of different wavelengths.

For example, the peak wavelength (the first peak wavelength) of a first light emitted from the first light-emitting element 11 is different from the peak wavelength (the second peak wavelength) of a second light emitted from the second light-emitting element 12. For example, in the first operation OP1, the first light-emitting element 11 is set to the light-emitting state; and the second light-emitting element 12 is set to the non-light-emitting state. Thereby, sensing is performed using the first peak wavelength. In the second operation OP2, the second light-emitting element 12 is set to the light-emitting state; and the first light-emitting element 11 is set to the non-light-emitting state. Thereby, sensing is performed using the second peak wavelength. The first light of the first peak wavelength is irradiated on the object independently from the second light of the second peak wavelength. Thereby, the characteristics that correspond to each peak wavelength can be sensed with high sensitivity. Thereby, high-precision sensing is possible. Such processing may be performed by the processor 70.

The processor 70 is provided in the embodiment as described above. The processor 70 processes the light reception signal obtained by the light receiving element 30. The processor 70 processes the first light reception signal obtained by the light receiving element 30 in the first operation OP1 and the second light reception signal obtained by the light receiving element 30 in the second operation OP2. Thereby, for example, high-precision sensing can be implemented using the characteristics corresponding to the different peak wavelengths.

Even in the case where the peak wavelength of emitted light is the same between the first light-emitting element 11 and the second light-emitting element 12, the processor 70 may process the first light reception signal obtained by the light receiving element 30 in the first operation OP1 and the second light reception signal obtained by the light receiving element 30 in the second operation OP2. The distance from the light receiving element 30 is different for the first light-emitting element 11 and the second light-emitting element 12. Therefore, information of the object at different depths is obtained by using these light-emitting elements. For example, better sensing can be implemented with high precision by performing the sensing using information relating to a blood vessel existing at a deep position (e.g., the subcutaneous plexus, etc.) and information relating to a blood vessel existing at a shallow position (e.g., the subpapillary plexus, etc.).

### Second Embodiment

In the description relating to the second embodiment recited below, a description is omitted for portions similar to those of the first embodiment. The description relating to the first embodiment is applicable appropriately to the second embodiment.

FIG. 8A and FIG. 8B are schematic views illustrating a sensor according to the second embodiment. FIG. 8A is a plan view. FIG. 8B is a line A1-A2 cross-sectional view of FIG. 8A.

As shown in FIG. 8A, the sensor 152 according to the embodiment includes the multiple light-emitting elements 10 and the multiple light receiving elements 30.

The multiple light-emitting elements 10 are arranged along the first direction (e.g., the X-axis direction) and along the second direction (e.g., the Y-axis direction) intersecting the first direction. The multiple light receiving elements 30 are arranged along the first direction and the second direction. At least a portion of the light L10 emitted from each of the multiple light-emitting elements 10 is incident on the multiple light receiving elements 30.

The multiple light-emitting elements 10 and the multiple light receiving elements 30 are arranged alternately along the first direction. In the example, the multiple light-emitting elements 10 and the multiple light receiving elements 30 are further arranged alternately along the second direction.

For example, the multiple light-emitting elements 10 include the first light-emitting element 11 and the second light-emitting element 12. On the other hand, the multiple light receiving elements 30 include a first light receiving element 31 and a second light receiving element 32. The second light-emitting element 12 is provided between the first light-emitting element 11 and the second light receiving element 32. The first light receiving element 31 is provided between the second light-emitting element 12 and the second light receiving element 32.

The multiple light-emitting elements 10 may further include the third light-emitting element 13. For example, the second light-emitting element 12 may be provided between the third light-emitting element 13 and the first light-emitting element 11. The third light-emitting element 13 may be provided between the first light-emitting element 11 and the second light-emitting element 12.

The multiple light receiving elements 30 may further include a third light receiving element 33. For example, the second light receiving element 32 may be provided between the third light receiving element 33 and the first light receiving element 31. The third light receiving element 33 may be provided between the first light receiving element 31 and the second light receiving element 32.

In the sensor 152 shown in FIG. 8B, the multiple light-emitting elements 10 and the multiple light receiving elements 30 are provided at the first surface 81 of the substrate 80. The light L10 that is emitted from the multiple light-emitting elements 10 is incident on the sensing object after passing through the substrate 80. The light that is reflected (scattered) by the sensing object is incident on the light receiving element 30.

The multiple light-emitting elements 10 are arranged in a matrix configuration. The multiple light receiving elements 30 also are arranged in a matrix configuration. For example, one or more of the multiple light-emitting elements 10 is set to the light-emitting state (the first state st1); and the light L10 that is emitted is sensed by one or more of the multiple light receiving elements 30. Thereby, any object (e.g., a blood vessel, etc.) can be sensed with high precision.

For example, one or more of the multiple light-emitting elements 10 is set to the light-emitting state (the first state st1) according to the configuration of the object (e.g., the blood vessel, etc.). Then, the sensing is performed using the light reception signal that is obtained by one or more of the multiple light receiving elements 30.

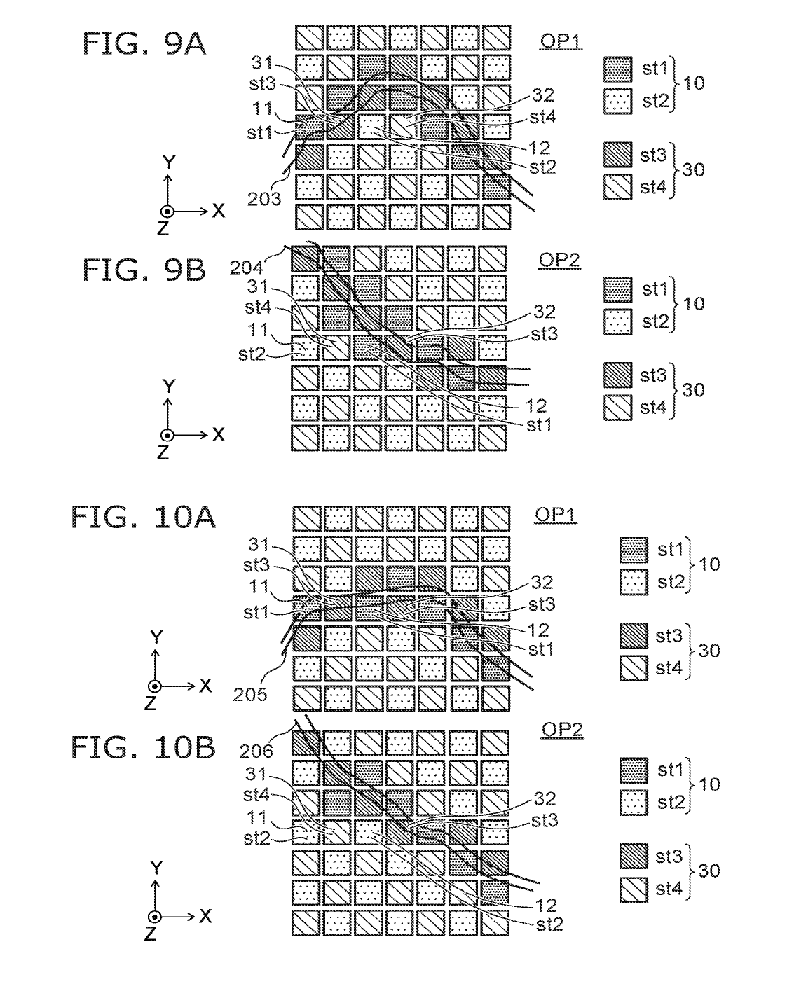

FIG. 9A and FIG. 9B are schematic plan views illustrating operations of a sensor according to a second embodiment.

FIG. 9A illustrates the first operation OP1 implemented by the sensor 152. FIG. 9B illustrates the second operation OP2 implemented by the sensor 152. The substrate 80, the controller 60, the processor 70, etc., are not shown in these drawings.

As shown in FIG. 9A, a blood vessel 203 is the object of the sensing in the first operation OP1. As shown in FIG. 9B, a blood vessel 204 is the object of the sensing in the second operation OP2. The configuration of the blood vessel 204 is different from the configuration of the blood vessel 203.

In these operations, a portion of the multiple light-emitting elements 10 is set to the first state st1 (the light-emitting state). Another portion of the multiple light-emitting elements 10 is set to the second state st2 (the non-light-emitting state).

In the example, the light-emitting elements 10 in the first state st1 are arranged along the configuration of the blood vessel 203 or 204. For example, the light-emitting elements 10 that overlap the blood vessel 203 or 204 in the Z-axis direction are set to the first state st1. For example, the light-emitting elements 10 that have a distance to the blood vessel 203 or 204 that is not more than a prescribed threshold are set to the first state st1. Then, the other light-emitting elements 10 are set to the second state st2.

Thus, for example, in the first operation OP1, the first light-emitting element 11 is in the first state st1 (the light-emitting state); and the second light-emitting element 12 is in the second state st2 (the non-light-emitting state). On the other hand, in the second operation OP2, the first light-emitting element 11 is in the second state st2 (the non-light-emitting state); and the second light-emitting element 12 is in the first state st1 (the light-emitting state). The state of the blood vessel 203 or 204 can be sensed with high precision by selectively setting a portion of the multiple light-emitting elements 10 to the light-emitting state according to the blood vessel 203 or 204.

On the other hand, the signal processing may be performed by selectively using the signals received by a portion of the multiple light receiving elements 30. For example, a portion of the multiple light receiving elements 30 are set to a third state st3 (a processing state). Then, another portion of the multiple light receiving elements 30 is set to a fourth state st4 (a non-processing state). For example, the light receiving elements 30 in the third state st3 are arranged along the configuration of the blood vessel 203 or 204. For example, the light receiving elements 30 that overlap the blood vessel 203 or 204 in the Z-axis direction are set to the third state st3.

For example, in the first operation OP1, the first light receiving element 31 is proximal to the blood vessel 203. For example, the first light receiving element 31 is set to the third state st3 (the processing state). In other words, the light reception signal that is obtained by the first light receiving element 31 is used in the processing. In the first operation OP1, the second light receiving element 32 is distal to the blood vessel 203. For example, the second light receiving element 32 is set to the fourth state st4 (the non-processing state). In other words, the light reception signal that is obtained by the second light receiving element 32 is not used in the processing.

In other words, in the first operation OP1, the distance between the first light-emitting element 11 and the blood vessel 203 is shorter than the distance between the second light-emitting element 12 and the blood vessel 203. The first light-emitting element 11 is in the first state st1 (the first light-emitting state); and the second light-emitting element 12 is in the second state st2 (the second non-light-emitting state). The distance between the first light receiving element 31 and the blood vessel 203 is shorter than the distance between the second light receiving element 32 and the blood vessel 203. The first light receiving element 31 is in the third state st3 (the processing state); and the second light receiving element 32 is in the fourth state st4 (the non-processing state).

On the other hand, in the second operation OP2, the distance between the first light-emitting element 11 and the blood vessel 204 is longer than the distance between the second light-emitting element 12 and the blood vessel 204. The first light-emitting element 11 is in the second state st2 (the first non-light-emitting state); and the second light-emitting element 12 is in the first state st1 (the first light-emitting state). The distance between the first light receiving element 31 and the blood vessel 204 is longer than the distance between the second light receiving element 32 and the blood vessel 204. The first light receiving element 31 is in the fourth state st4 (the non-processing state); and the second light receiving element 32 is in the third state st3 (the processing state).

In the embodiment, for example, the information of the blood vessel can be sensed with high precision by setting the light-emitting elements 10 arranged along the configuration of the blood vessel to the first state st1 and by further setting the light receiving elements 30 arranged along the configuration of the blood vessel to the third state st3.

Such operations may be implemented by the controller 60 and the processor 70.

For example, in the embodiment, the controller 60 that controls the operations of the multiple light-emitting elements 10 is provided. The multiple light-emitting elements 10 include the first light-emitting element 11 and the second light-emitting element 12. In such a case, the controller 60 implements the first operation OP1 and the second operation OP2. In the first operation OP1, the first light-emitting element 11 is in the first light-emitting state (the first state st1); and the second light-emitting element 12 is in the second non-light-emitting state (the second state st2). In the second operation OP2, the first light-emitting element 11 is in the first non-light-emitting state (the second state st2); and the second light-emitting element 12 is in the second light-emitting state (the first state st1).

In the embodiment, the processor 70 that processes the light reception signals obtained by the multiple light receiving elements 30 is provided. The multiple light receiving elements 30 include the first light receiving element 31 and the second light receiving element 32. In such a case, for the processor 70 in the first operation OP1, the first light receiving element 31 is in the third state st3; and the second light receiving element 32 is in the fourth state st4. In other words, in the first operation OP1, the processor 70 processes the light reception signal obtained by the first light receiving element 31 but does not process the light reception signal obtained by the second light receiving element 32. For the processor 70 in the second operation OP2, the first light receiving element 31 is in the fourth state st4; and the second light receiving element 32 is in the third state st3. In other words, in the second operation OP2, the processor 70 processes the light reception signal obtained by the second light receiving element 32 without processing the light reception signal obtained by the first light receiving element 31.

For example, such operations may be performed by implementing the pre-operation described in reference to FIG. 3. For example, the pre-operation is implemented prior to the sensing of the object.

FIG. 10A and FIG. 10B are schematic plan views illustrating operations of the sensor according to the second embodiment.

FIG. 10A illustrates the first operation OP1 implemented by the sensor 152. FIG. 10B illustrates the second operation OP2 implemented by the sensor 152. The substrate 80, the controller 60, the processor 70, etc., are not shown in these drawings.

In the first operation OP1 shown in FIG. 10A, a blood vessel 205 is the object of the sensing. The first light-emitting element 11 is in the first state st1 (the first light-emitting state); and the second light-emitting element 12 also is in the first state st1 (the second light-emitting state). The first light receiving element 31 is in the third state st3 (the processing state); and the second light receiving element 32 also is in the third state st3 (the processing state).

In the second operation OP2 shown in FIG. 10B, a blood vessel 206 is the object of the sensing. The first light-emitting element 11 is in the second state st2 (the first non-light-emitting state); and the second light-emitting element 12 also is in the second state st2 (the second non-light-emitting state). The first light receiving element 31 is in the fourth state st4 (the non-processing state); and the second light receiving element 32 is in the third state st3 (the processing state).

In the example, among the multiple light-emitting elements 10, the second light-emitting element 12 is most proximal to the first light-emitting element 11. In the embodiment, the position of the second light-emitting element 12 relative to the first light-emitting element 11 is arbitrary. Among the multiple light receiving elements 30, the second light receiving element 32 is most proximal to the first light receiving element 31. In the embodiment, the position of the second light receiving element 32 relative to the first light receiving element 31 is arbitrary.

In the example recited above, the light-emitting elements 10 that are proximal to the blood vessel are set to the first state st1 (the light-emitting state); and the light-emitting elements 10 that are distal to the blood vessel are set to the second state st2 (the non-light-emitting state). Such operations may be implemented in the case where the position of the blood vessel is shallow. On the other hand, in the case where the position of the blood vessel is deep, the light-emitting elements 10 that are distal to the blood vessel may be set to the first state st1 (the light-emitting state); and the light-emitting elements 10 that are proximal to the blood vessel may be set to the second state st2 (the non-light-emitting state). Thereby, high-precision sensing can be performed for a deep blood vessel. An example of such a case will now be described.

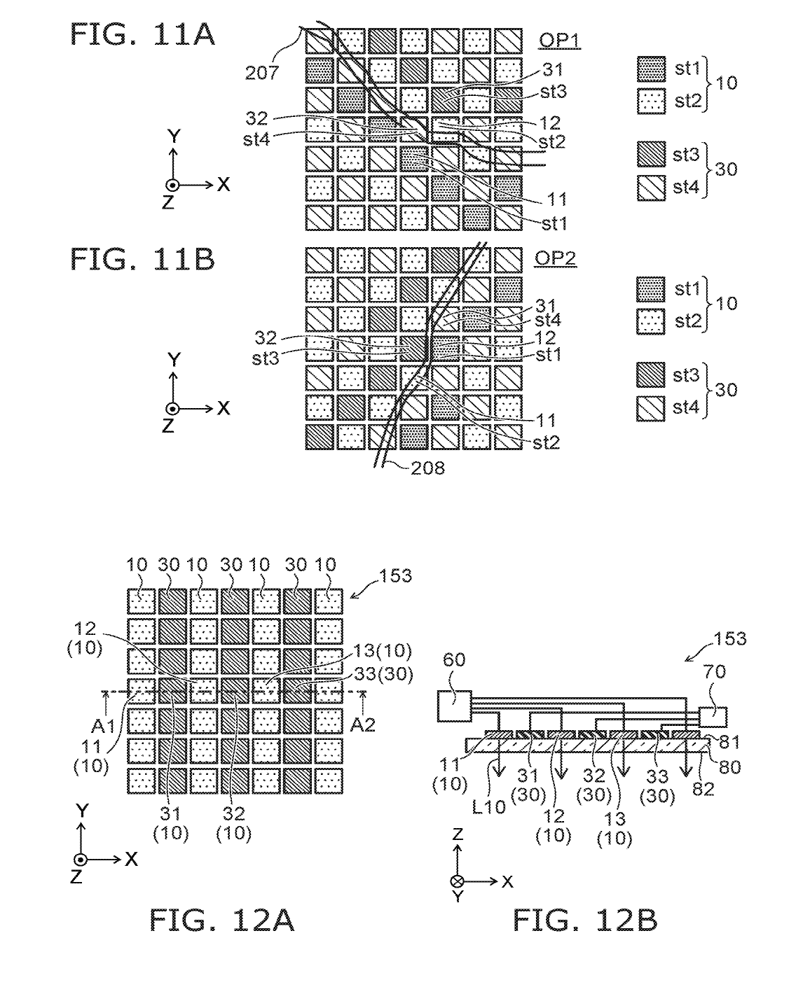

FIG. 11A and FIG. 11B are schematic plan views illustrating other operations of the sensor according to the second embodiment.

FIG. 11A illustrates another first operation OP1 implemented by the sensor 152. FIG. 11B illustrates another second operation OP2 implemented by the sensor 152. The substrate 80, the controller 60, the processor 70, etc., are not shown in these drawings.

In the first operation OP1 shown in FIG. 11A, a blood vessel 207 is the object of the sensing. The distance between the first light-emitting element 11 and the blood vessel 207 is longer than the distance between the second light-emitting element 12 and the blood vessel 207. The first light-emitting element 11 that is distal to the blood vessel 207 is in the first state st1 (the first light-emitting state); and the second light-emitting element 12 that is proximal to the blood vessel 207 is in the second state st2 (the first non-light-emitting state). The distance between the first light receiving element 31 and the blood vessel 207 is longer than the distance between the second light receiving element 32 and the blood vessel 207. The first light receiving element 31 that is distal to the blood vessel 207 is in the third state st3 (the processing state); and the second light receiving element 32 that is proximal to the blood vessel 207 is in the fourth state st4 (the non-processing state).

In the second operation OP2 shown in FIG. 11B, a blood vessel 208 is the object of the sensing. The distance between the first light-emitting element 11 and the blood vessel 208 is shorter than the distance between the second light-emitting element 12 and the blood vessel 208. The first light-emitting element 11 that is proximal to the blood vessel 208 is in the second state st2 (the first non-light-emitting state); and the second light-emitting element 12 that is distal to the blood vessel 208 is in the first state st1 (the first light-emitting state). The distance between the first light receiving element 31 and the blood vessel 208 is shorter than the distance between the second light receiving element 32 and the blood vessel 208. The first light receiving element 31 that is proximal to the blood vessel 208 is in the fourth state st4 (the non-processing state); and the second light receiving element 32 that is distal to the blood vessel 208 is in the third state st3 (the processing state).

In the example shown in FIG. 11A and FIG. 11B, one of the multiple light-emitting elements 10 in one of two regions divided by the blood vessel 207 or 208 when projected onto the X-Y plane is set to the first state st1; and one of the multiple light receiving elements 30 in the other of the two regions is set to the third state st3. The light-emitting elements 10 in the first state st1 are arranged along the blood vessel. The light receiving elements 30 in the third state st3 are arranged along the blood vessel.

For example, an operation (e.g., the first operation) of setting the light-emitting elements 10 proximal to the blood vessel to the first state st1 (the light-emitting state) and setting the light-emitting elements 10 distal to the blood vessel to the second state st2 (the non-light-emitting state) may be implemented; and an operation (e.g., the second operation) of setting the light-emitting elements 10 distal to the blood vessel to the first state st1 (the light-emitting state) and setting the light-emitting elements 10 proximal to the blood vessel to the second state st2 (the non-light-emitting state) may be implemented. Thereby, information is obtained for the shallow blood vessel and the deep blood vessel. Sensing with higher precision is possible by using such information for the multiple blood vessels. For example, the processing can be implemented by the processor 70.

In other words, the processor 70 that processes the light reception signals obtained by the multiple light receiving elements 30 is provided in the sensor 152. The multiple light receiving elements 30 include the first light receiving element 31 and the second light receiving element 32. The processor 70 processes the first light reception signal obtained by the first light receiving element 31 and the second light reception signal obtained by the second light receiving element 32.

In the embodiment as well, the peak wavelengths of the light emitted by each of two of the multiple light-emitting elements 10 may be different from each other. The object of the sensing (e.g., the blood vessel) is sensed using the two or more light-emitting elements 10 having different peak wavelengths. For example, information relating to the oxygen concentration in the blood can be obtained. The applications of the sensor are expanded by using light of different wavelengths.

FIG. 12A and FIG. 12B are schematic views illustrating another sensor according to the second embodiment.

FIG. 12A is a plan view. FIG. 12B is a line A1-A2 cross-sectional view of FIG. 12A.

As shown in FIG. 12A, the sensor 153 according to the embodiment includes the multiple light-emitting elements 10 and the multiple light receiving elements 30.

The multiple light-emitting elements 10 are arranged along the first direction (e.g., the X-axis direction) and along the second direction (e.g., the Y-axis direction) intersecting the first direction. The multiple light receiving elements 30 are arranged along the first direction and the second direction. At least a portion of the light L10 emitted from each of the multiple light-emitting elements 10 is incident on the multiple light receiving elements 30.

The multiple light-emitting elements 10 and the multiple light receiving elements 30 are arranged alternately along the first direction (e.g., the X-axis direction).

The multiple light-emitting elements 10 are arranged in the X-Y plane. The multiple light receiving elements 30 also are arranged in the X-Y plane. For example, one or more of the multiple light-emitting elements 10 are set to the light-emitting state (the first state st1); and the light L10 that is emitted is sensed by one or more of the multiple light receiving elements 30. Thereby, any object (e.g., a blood vessel, etc.) can be sensed with high precision. For example, one or more of the multiple light-emitting elements 10 is set to the light-emitting state (the first state st1) according to the configuration of the object (e.g., the blood vessel, etc.). Then, the sensing is performed using the light reception signal obtained by the one or more of the multiple light receiving elements 30. In the sensor 153 as well, high-precision sensing can be implemented.

### Third Embodiment

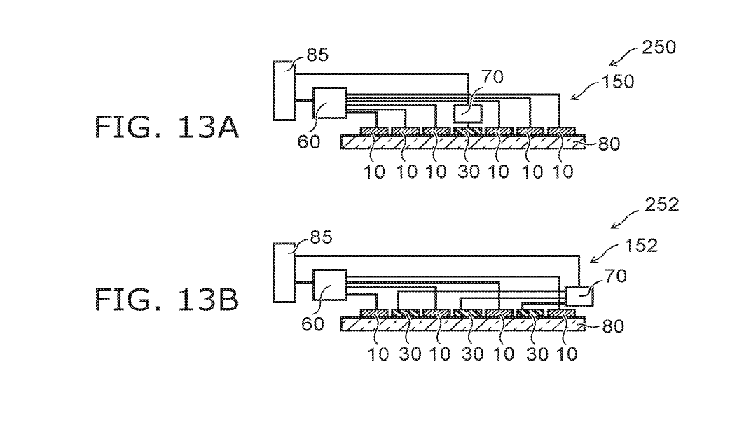

FIG. 13A and FIG. 13B are schematic views illustrating sensor systems according to a third embodiment.

As shown in FIG. 13A, a sensor system 250 according to the embodiment includes the sensor 150 and an interface unit 85. The interface unit 85 supplies the sense signal sensed by the sensor 150 to the outside. The interface unit 85 may acquire a control signal from the outside and supply the control signal to at least one of the controller 60 or the processor 70.

As shown in FIG. 13B, a sensor system 252 according to the embodiment includes the sensor 152 and the interface unit 85. The sensors according to the first and second embodiments and modifications of the first and second embodiments may be used as the sensor of the embodiment.

For example, the sensors according to the first and second embodiments recited above and the sensor system according to the third embodiment are applicable to the sensing of the pulse wave of a living body.

For example, in the field of medicine, the pulse waveform (e.g., the pulse waveform of an artery) is measured. For example, the analysis of the pulse wave is performed in an examination of the circulatory system (e.g., an arteriosclerosis level measurement or a stress level measurement). For example, the analysis of the pulse wave is performed also by a pulse oximeter (arterial oxygen saturation measuring device).

For example, technology is being developed to constantly measure the pulse wave by using portable measuring devices such as a wristwatch-type device, a device adhered to the living body, etc. For example, in photoplethysmography, the waveform of the pulse wave is measured percutaneously without paracentesis or drawing blood. In such a method, it is possible to suppress the burden on the living body and perform the measurement easily. Therefore, there are expectations for a wide range of applications in the field of health care. For example, the blood pressure is estimated by calculating the acceleration pulse wave from the waveform of the pulse wave and analyzing the characteristic points of the acceleration pulse wave.

For example, in the field of medicine, a photoplethysmograph measuring device is mounted to a finger tip or an ear lobe; light is irradiated on the living body; and the light that passes through the living body is sensed. On the other hand, the photoplethysmograph measuring device that is constantly mounted to the finger tip or the ear lobe is inappropriate for applications in the field of health care because the burden is large. Wristwatch-type measuring devices are being developed from this perspective. However, compared to a finger tip or an ear lobe, the density of the blood vessels is low and the signal of the pulse wave is weak for the wrist, the chest, etc. If the density of the blood vessels is low, it is difficult to measure the waveform of the pulse wave with high precision.

In the embodiments recited above, the pulse wave can be sensed with high precision at the wrist, the chest, etc., where the blood vessel density is low. Because multiple light-emitting elements are provided, the load of one light-emitting element can be reduced.

In the embodiments, the multiple light-emitting elements are arranged in a lattice configuration. The multiple light-emitting elements emit, onto a measurement region of at least a portion of the living body, at least one type of measurement light belonging to a prescribed wavelength band. A light receiving element is provided. The measurement light that is emitted from the multiple light-emitting elements and passes through the living body is sensed by the light receiving element. For example, as the multiple light-emitting elements, an array of light sources (light-emitting elements) such as OLEDs, etc., are used. Such a configuration is less expensive than using a light receiving element array having a large surface area. The drive circuit is simpler for an array of light-emitting elements than for an array of light receiving elements. Low noise and a high S/N ratio are obtained by using OLEDs. In such a case, the driving time and luminance of one light source can be suppressed by using the array of OLED light sources. Thereby, a long life is obtained.

In the sensing method according to the embodiments, for example, the sensing is performed using multiple light sources (light-emitting elements) and a light receiving element. The multiple light sources emit, onto the measurement region of at least a portion of the living body, at least one type of measurement light belonging to a prescribed wavelength band. The multiple light sources are arranged in a lattice configuration. The measurement light that is emitted from the multiple light sources and passes through the living body is sensed by the light receiving element. In the sensing method, the analysis processing is performed based on the temporal change of the light amount of the measurement light that is sensed. The analysis processing includes designating the measurement position inside the measurement region. The measurement position is used to measure information relating to the pulsatory motion accompanying the activity of the living body.

A program according to the embodiments causes a computer to implement the sensing method recited above. The program recited above is recorded in a recording medium according to the embodiments.

According to the embodiments, a sensor and a sensor system are provided in which the sensing precision can be increased.

In the specification of the application, “perpendicular” and “parallel” refer to not only strictly perpendicular and strictly parallel but also include, for example, the fluctuation due to manufacturing processes, etc. It is sufficient to be substantially perpendicular and substantially parallel.

Hereinabove, exemplary embodiments of the invention are described with reference to specific examples. However, the embodiments of the invention are not limited to these specific examples. For example, one skilled in the art may similarly practice the invention by appropriately selecting specific configurations of components included in sensors such as light emitting layers, light receiving elements, substrates, organic light emitting layers, organic photoelectric conversion layers, electrodes, controllers, processors, etc., from known art. Such practice is included in the scope of the invention to the extent that similar effects thereto are obtained.

Further, any two or more components of the specific examples may be combined within the extent of technical feasibility and are included in the scope of the invention to the extent that the purport of the invention is included.

Moreover, all sensors and sensor systems practicable by an appropriate design modification by one skilled in the art based on the sensors and sensor systems described above as embodiments of the invention also are within the scope of the invention to the extent that the spirit of the invention is included.

Various other variations and modifications can be conceived by those skilled in the art within the spirit of the invention, and it is understood that such variations and modifications are also encompassed within the scope of the invention.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the invention.

What is claimed is:

1. A sensor, comprising:
a plurality of light-emitting elements arranged along a first direction and a second direction, the second direction intersecting the first direction; and
a light receiving element, at least a portion of light emitted from each of the light-emitting elements being incident on the light receiving element,
the light-emitting elements not overlapping the light receiving element in a third direction, the third direction being perpendicular to the first direction and the second direction,
the light-emitting elements including a first light-emitting element and a second light-emitting element,
a position of the second light-emitting element in a plane being between a position of the first light-emitting element in the plane and a position of the light receiving element in the plane, the plane including the first direction and the second direction.

2. The sensor according to claim 1, wherein
the light-emitting elements further includes a third light-emitting element and a fourth light-emitting element, and
a position of the fourth light-emitting element in the plane is between a position of the third light-emitting element in the plane and the position of the light receiving element in the plane.

3. The sensor according to claim 1, wherein the light-emitting elements are provided around the light receiving element in the plane.

4. The sensor according to claim 3, further comprising a substrate,
the substrate having a first surface and a second surface, the first surface being along the first direction and the second direction, the second surface being on a side opposite to the first surface,
the light-emitting elements being provided at the first surface,
the light receiving element being provided at one of the first surface or the second surface.

5. The sensor according to claim 1, further comprising a substrate,
the substrate having a first surface along the first direction and the second direction,
the first surface including a first region and a second region, the second region being on an outer side of the first region,
the light-emitting elements overlapping the first region in a third direction, the third direction being perpendicular to the first direction and the second direction,
the light receiving element overlapping the second region in the third direction.

6. The sensor according to claim 5, wherein
the substrate further has a second surface on a side opposite to the first surface,
the light-emitting elements are provided at the first surface, and
the light receiving element is provided at one of the first surface or the second surface.

7. The sensor according to claim 1, wherein a peak wavelength of a first light emitted from the first light-emitting element is different from a peak wavelength of a second light emitted from the second light-emitting element.

8. The sensor according to claim 1, further comprising a controller to control operations of the light-emitting elements,
wherein the controller implements a first operation and a second operation,
in the first operation, the first light-emitting element is in a first light-emitting state, and the second light-emitting element is in a second non-light-emitting state,
in the second operation, the first light-emitting element is in a first non-light-emitting state, and the second light-emitting element is in a second light-emitting state.

9. The sensor according to claim 1, further comprising a controller to control operations of the light-emitting elements,
wherein the controller implements a pre-operation of setting each of the light-emitting elements to a light-emitting state,
the controller implements a first operation after the pre-operation,
in the first operation, the first light-emitting element determined based on a result of the pre-operation is in a first light-emitting state, and the second light-emitting element determined based on the result of the pre-operation is in a second non-light-emitting state.

10. The sensor according to claim 9, further comprising a processor to process light reception signals obtained by the light receiving element,
wherein the processor processes a first light reception signal and a second light reception signal, the first light reception signal is obtained by the light receiving element in the first operation, the second light reception signal is obtained by the light receiving element in the second operation.

11. A sensor, comprising:
a plurality of light-emitting elements arranged along a first direction and a second direction, the second direction intersecting the first direction; and
a plurality of light receiving elements arranged along the first direction and the second direction, at least a portion of light emitted from each of the light-emitting elements being incident on the light receiving elements,
the light-emitting elements and the light receiving elements being arranged alternately along the first direction.

12. The sensor according to claim 11, wherein the light-emitting elements and the light receiving elements are further arranged alternately along the second direction.

13. The sensor according to claim 11, further comprising a controller to control operations of the light-emitting elements,
wherein
the light-emitting elements include a first light-emitting element and a second light-emitting element,
the controller implements a first operation and a second operation,
in the first operation, the first light-emitting element is in a first light-emitting state, and the second light-emitting element is in a second non-light-emitting state,
in the second operation, the first light-emitting element is in a first non-light-emitting state, and the second light-emitting element is in a second light-emitting state.

14. The sensor according to claim 11, further comprising a controller to control operations of the light-emitting elements,
wherein
the light-emitting elements include a first light-emitting element and a second light-emitting element,
the controller implements a first operation and a second operation,
in the first operation, the first light-emitting element is in a first light-emitting state, and the second light-emitting element is in a second light-emitting state,
in the second operation, the first light-emitting element is in a first non-light-emitting state, and the second light-emitting element is in a second non-light-emitting state.

15. The sensor according to claim 14, wherein the second light-emitting element is most proximal to the first light-emitting element among the light-emitting elements.

16. The sensor according to claim 11, further comprising a processor to process light reception signals obtained by the light receiving elements,
wherein
the light receiving elements include a first light receiving element and a second light receiving element,
the processor processes a first light reception signal and a second light reception signal, the first light reception signal is obtained by the first light receiving element, the second light reception signal is obtained by the second light receiving element.

17. The sensor according to claim 11, wherein peak wavelengths of light emitted from two of the light-emitting elements are different from each other.

18. The sensor according to claim 11, wherein each of the light receiving elements includes an organic photoelectric conversion layer.

19. The sensor according to claim 11, wherein each of the light emitting elements includes an organic light emitting layer.

20. A sensor system comprising:
a sensor; and
an interface unit supplying a sense signal sensed by the sensor to outside,
the sensor including
a plurality of light-emitting elements arranged along a first direction and a second direction, the second direction intersecting the first direction, and
a light receiving element, at least a portion of light emitted from each of the light-emitting elements being incident on the light receiving element,
the light-emitting elements not overlapping the light receiving element in a third direction, the third direction being perpendicular to the first direction and the second direction,
the light-emitting elements including a first light-emitting element and a second light-emitting element,
a position of the second light-emitting element in a plane being between a position of the first light-emitting element in the plane and a position of the light receiving element in the plane, the plane including the first direction and the second direction.
